(19) United States
(12) Patent Application Publication
(10) Pub. No.: US 2008/0242932 A1
(43) Pub. Date: Oct. 2, 2008

(54) Endoscopic Securing System

(75) Inventor: Matthew P. Carter, Dobson, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/056,918

(22) Filed: Mar. 27, 2008

Related U.S. Application Data
(60) Provisional application No. 60/920,855, filed on Mar. 30, 2007.

(51) Int. Cl. A61B 1/00

(52) U.S. Cl. 600/127

(57) ABSTRACT

An endoscopic securing system for use with a cap member disposed on the distal end of an endoscope, the securing system including a tether and a safety coupling. The safety coupling is attached around the body of an endoscope and is coupled to the cap member by the tether to prevent distal movement or dislodgment of the cap member. When the system is placed onto an endoscope, the tethers extend axially along the endoscope to the safety coupling. The clamp can also be disposed proximally adjacent to a ridge on the endoscope to prevent the safety coupling from moving distally toward the cap member.

### CROSS REFERENCE TO RELATED APPLICATION

This application claims priority to U.S. Provisional Application No. 60/920,855, filed on Mar. 30, 2007, the entire contents of which are hereby incorporated by reference.

### TECHNICAL FIELD

The present invention relates to an improved endoscopic securing system having safety features to secure a cap member to the distal end of an endoscope and prevent the dislodgement of a cap member from an endoscope.

### BACKGROUND

The treatment of tissue encompasses a variety of techniques such as electrocauterization, heat therapy, resection (removal of tissue), and sclerotherapy (the injection of medicine into target tissue). These treatment techniques usually involve the passing of medical instruments through the operating channel of the endoscope. The endoscope permits minimally invasive access, as well as visualization and suction aids.

Another technique that frequently utilizes the operating channel of the endoscope is ligation, which involves applying a band or ligature around a vessel or portion of tissue, thereby cutting off blood or fluid flow and causing the tissue to necrose and separate from adjacent healthy tissue. Ligation is widely used to treat a number of medical tissue conditions, including, but not limited to, hemorrhoids, polyps, ballooning varices, and other types of lesions, including those that are cancerous. Typically, ligators are also used with a suction or vacuum means to draw the tissue into the distal end, whereby the band is deployed over the base of the diseased tissue to cut off blood flow. The ligating device is typically activated by retracting a line (string, wire, or cable) that is attached to the ligator at the distal end of an endoscope and is threaded through the operating channel of the endoscope to the proximal end of the instrument. The ligator can be activated by mechanically pulling the activating line by means of a hand-operated reel or trigger, or by a motor drive mechanism. Various other ligating devices use cooperating inner and outer members that slide the individual bands by pushing or pulling them from the end of the inner or outer member, the bands being preloaded onto the inner or outer member prior to deployment.

A ligator device can become dislodged during an endoscopic procedure. The ligator is typically placed on the end of an endoscope covering the cap. Due to the sometimes vigorous movements of the endoscope, the ligator can become dislodged and separated from the endoscope. To recover the ligator, a physician will typically have to withdraw the endoscope and then re-enter with a grasping device, such as forceps, to retrieve the ligator. This retrieval delays the surgical procedure and places the patient at greater risk. Therefore, there is a need for improved ligator devices with safety features that prevent their dislodgement and/or separation from an endoscope cap. There is also a need for a means of efficiently retrieving a device that has become dislodged and/or separated from the endoscope.

### BRIEF SUMMARY

Herein provided is an endoscopic securing system for devices used in the endoscopic treatment of tissue. The system comprises a cap member configured to be disposed on the distal end of an endoscope, a safety harness, and a safety coupling. The safety coupling is adapted to be securable to the endoscope between the proximal end of the endoscope and the cap member. The safety coupling is formed to fit around the endoscope and the safety harness is connected to the cap member and extends in a proximal direction to connect with the safety coupling. In one embodiment of the present invention, the securing system further comprises an endoscope with a ridge distally adjacent to the safety coupling that prevents the safety coupling from moving distally toward the distal end of the endoscope. In other embodiments, the safety harness comprises one of at least one tether or a securing sleeve.

The cap member can be a ligating barrel, sectomy hood, or any other endoscopic device that can be attached to the distal end of an endoscope. If a ligating barrel is used, there can be one or more ligating bands removably disposed on the barrel. In other embodiments of the invention, there can be two tethers connecting the cap member and the safety coupling. The tethers can extend through the safety coupling to a point near the proximate end of the endoscope so that they can be coupled to an activation device.

The endoscopic securing system secures the cap member to the distal end of an endoscope. The method comprises providing a cap member disposed on the distal end of an endoscope, a safety harness connected to the cap member and extending in a proximal direction from the cap member, and coupled to a safety coupling. The safety coupling is secured to the endoscope at a location proximal to and apart from the cap member. Then the safety harness is extended from the cap member in a proximal direction approaching the safety coupling.

The endoscopic securing system is also well suited for use in methods of retrieving a dislodged cap member by using an actuating device to pull the tether proximally. The cap member can be retrieved by withdrawing the endoscope in some embodiments.

### BRIEF DESCRIPTION OF THE DRAWINGS

FIG. 1 is a perspective view of the endoscopic securing system of the present invention.

FIG. 2 is a perspective view of the endoscopic securing system disposed on an endoscope.

FIG. 3 presents a side view and a cross sectional view of a safety coupling of the present invention. FIG. 3A is a cross sectional view of a safety coupling.

FIG. 4 is a cross sectional view showing auxiliary access channels for auxiliary endoscopic instruments.

FIG. 5 is a perspective view of a securing sleeve disposed on an endoscope.

### DETAILED DESCRIPTION OF THE DRAWINGS AND THE PRESENTLY PREFERRED EMBODIMENTS

The present invention provides a securing system for use with cap members adapted to be disposed on the distal end of an endoscope. At least one tether extends from the cap member and along the endoscope to a safety coupling. The safety harness is disposed around the endoscope at a location between the proximal end of the endoscope and the cap member. This location can vary depending on the length and type of safety harness used. A ridge can be disposed on the endoscope that prevents the safety coupling from sliding toward the distal end of the endoscope. The safety harness secures the cap member on the distal end of the endoscope and can be used to retrieve a cap member that becomes dislodged from the distal end of the endoscope. The safety harness is characteristically taut to assist in securing and stabilizing the cap member on the distal end of the endoscope. The safety coupling has a fastening mechanism such that it has a secure fit around the shaft of the endoscope thereby preventing the coupling from sliding distally. The inner surface of the safety coupling can be textured or comprise an adhesive or other means to provide frictional engagement with the outer surface of the endoscope.

In some embodiments, the most proximal end of the safety harness never enters the patient such that an operator may actuate the safety harness to retrieve the cap member. In other embodiments, the proximal end of the safety harness terminates at the safety coupling and typically enters the GI tract. The cap member can comprise any removable device attached to the scope to help provide a particular diagnostic or therapeutic function. The cap member can be, for example, a ligating barrel, endoscope hood, or other device that attaches to the distal end of an endoscope. Embodiments that may be preferred comprise cap members adapted to receive the proximal end of the safety harness. However, the safety harness of the present invention may be secured to current cap members known in the art.

Because the cap member is attached to the safety coupling by a safety harness, the likelihood that the cap member becomes dislodged is greatly decreased. In instances where the cap member becomes dislodged from the distal end of the endoscope, the securing system prevents the cap member from becoming lost in the body. In other embodiments, however, the safety harness is sufficiently flaccid to allow the cap member to move freely as the endoscope is maneuvered throughout the body. The safety harness can be at least one tether, elastic member (band, cord, or the like), spring, or a securing sleeve. The safety harness can be made of a resilient material that places tension on the cap member when the safety coupling is appropriately spaced. The tether can be made of synthetic fiber, string, wire, cable, or other suitable biocompatible material. The tether can be connected to any point on the safety coupling that provides secure attachment. In other embodiments, the tether proceeds through safety coupling channels to an actuation device. The length of the tether from the cap member to the safety coupling can be predetermined based on the procedure and patient. In some embodiments the length of the tether(s) can be from about 2 cm to about 20 cm or any length therein. The length of the tether(s) from the safety coupling to the proximal end of the endoscope can vary depending on the procedure and the particular scope used. The tether lengths from the safety coupling to the proximal end can be from about 100 cm to about 200 cm or any length therein. Such tether length(s) will be long enough to extend from the safety coupling to the proximal end of the endoscope and, in some embodiments, long enough to attach to an actuation device.

The securing sleeve is connected to the cap member and covers a portion of the endoscope from the cap member and extends in a proximal direction over the endoscope. The securing sleeve is generally tubular in shape and is comprised of material suitable for providing frictional engagement with the endoscope. The securing sleeve is deployable over the body of the endoscope to provide a friction fit with the endoscope. In some embodiments the securing sleeve is deployable by unfurling or unrolling the sleeve over the body of the endoscope in a proximal direction away from the cap member. As such, the present invention also provides an endoscopic securing system comprising a cap member, as described above, connected to a tubular safety harness, such as a securing sleeve, to prevent dislodgment of the cap member extending in a proximal direction from the cap member. In such embodiments, the safety coupling may or may not be used.

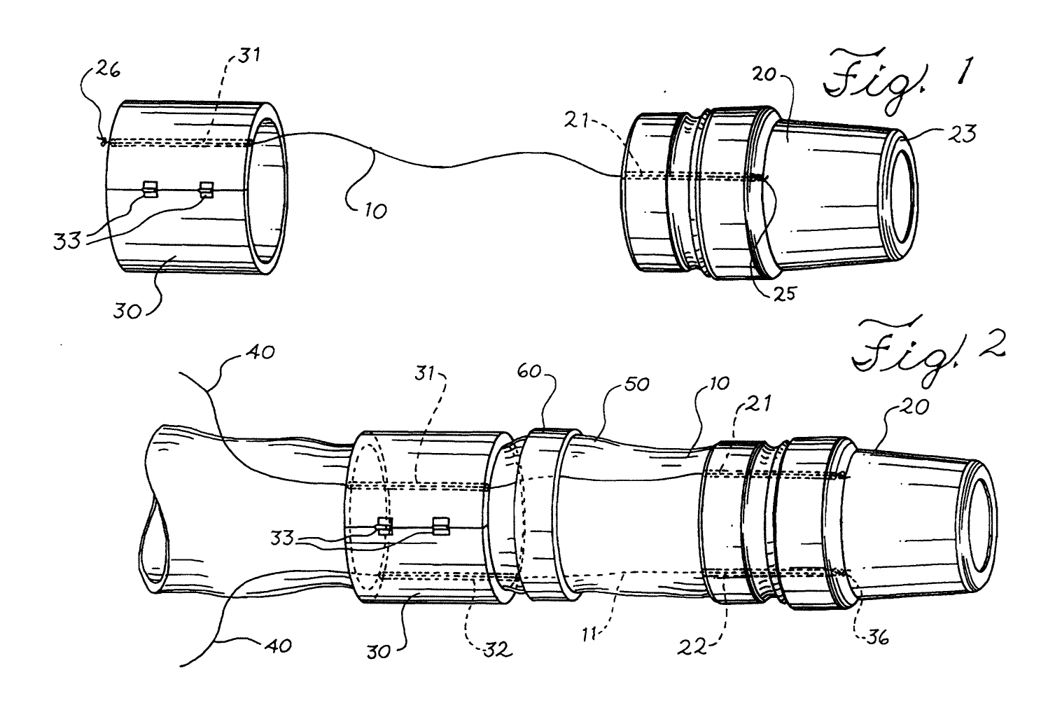

In some embodiments, the tether 10 can be movably attached to the safety coupling 30 while terminating in a cap member 20. As shown in FIG. 1, the tether 10 is connected to the cap member 20 by threading the tether 10 through a first channel 21 and tying a knot 25 at the distal opening 23 of the first channel 21 to prevent the tether 10 from decoupling. Although the tether 10 is connected to the safety coupling 30 and cap member 20, in this example by knots, it is understood that the tether 10 can be connected by other means sufficient to secure attachment of the tether 10 to the safety coupling 30 and/or cap member 20. For example, the tether may be attachable by cinching or clamping. The tether 10 can also be coupled to the safety coupling 30 by threading the line 10 through a first safety coupling channel 31 and tying a knot 26 at the proximal end of the channel 31. If the cap member 20 is dislodged from the distal end of the endoscope 50, a surgeon can recover the cap member 20 by withdrawing the endoscope 50 and reattaching the cap member 20 onto the distal end of the endoscope 50. Once reattached, the surgeon can reinsert the cap member 20 without a significant loss of operating time. There are also embodiments comprising one tether 10 or, such as in FIG. 2, at least two tethers 10 and 11 extending from the cap member 20 and terminating at the safety coupling 30.

In the embodiment shown in FIG. 2, a second tether 11 is threaded through a second cap member channel 22 and subsequently coupled by a knot 36 tied at the distal end of the second cap member channel 22. The second tether 11 extends axially along the endoscope 50 to a second safety coupling channel 32. The tethers 10 and 11, as shown in FIG. 2, are tightly drawn to secure the cap member 20 to the distal end of the endoscope 50. This decreases the likelihood of dislodgment. In some embodiments, the proximate end of the second tether 11 can be coupled to the safety coupling 30 by way of a knot (not shown), or the like, in the proximate end of the first 31 and second 32 safety coupling channels. Distally adjacent to the safety coupling 30 is a ridge 60 on the endoscope 50 that prevents the safety coupling 30 from sliding distally in the direction of the cap member 20. This ridge 60 is present on many types of endoscopes 50 and is formed by the proximal end of a flexible sheath that is disposed over the distal most portion of the endoscope 50. A dislodged cap member in this embodiment can be retrieved and reattached as previously discussed.

FIG. 2 shows an embodiment of the present invention where the tethers 10 and 11 extend from the cap member 20 through the safety coupling 30 without being affixed thereto. There can be embodiments where the tethers 10 and 11 are affixed or otherwise coupled to the safety coupling 30 but do not extend through channels 31 and 32 in the safety coupling. The proximate ends 40 of the tethers 10 and 11 preferably terminate at an actuation device at or near the proximate end of the endoscope 50. The proximate ends 40 can be attached to an actuation device such as a hand operated reel, trigger, or motor driven mechanism. The tethers 10 and 11 can also be actuated using the hand of a surgeon. Some embodiments have one tether or more than two tethers extending through the safety coupling 30 to the proximate end of the endoscope. Although shown with safety channels 10 and 11 in this example, multiple safety channels can be used with accompanying tethers.

In embodiments where the tethers extend to the proximate end of the endoscope 50, the proximate ends 40 of the tethers can be manipulated by the operator in the event the cap member 20 becomes dislodged from the distal end of the endoscope. Also, in such embodiments, there can be one or more proximate ends 40 extending from the safety coupling 30 to the proximate end of the endoscope 50. The proximate ends 40 can be attached to an actuation device so that an operator can pull the proximate ends 40 of the tethers 10 and 11 to retrieve the cap member 20 in the case of dislodgement. In such an event, the operator pulls proximally on the proximate ends 40 to move the safety coupling 30 in a proximal direction. By doing so, the cap member 20 is also moved in a proximal direction. The operator can then retrieve the cap member 20 by withdrawing the endoscope 50 fully from the body. In some embodiments, the operator can be capable of reattaching the cap member 20 on the distal end of the endoscope without withdrawing the endoscope from the body.

Cap members such as ligating barrels are suitable for this invention and are known in the art. A more detailed description of such barrels can be found in U.S. Pat. No. 5,624,453, incorporated herein by reference in its entirety. The ligating barrels can have one or more ligating bands removably disposed on the barrel. Endoscope hoods are also suitable cap members useful in some embodiments of the present invention. The securing system of the present invention can be used with any cap member adapted to fit on the distal end of an endoscope and used in endoscopic procedures.

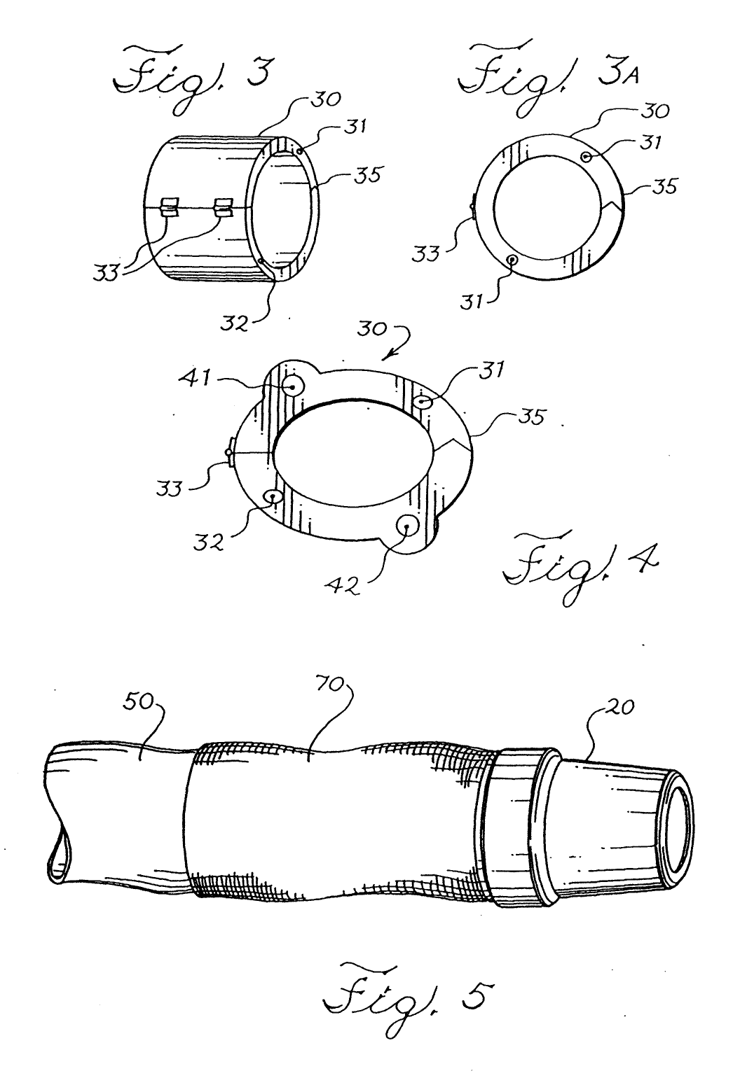

The safety coupling 30 can be made of a resilient material such as thermoplastics, polyurethanes, polycarbonates, or other polymers or metals. As shown in FIG. 3, the safety coupling 30 resembles a ring or a circular clamp that is attached to the endoscope with a fastening mechanism such as a clasp, buckle, friction fit, strap, or a snap fit 35, with a projection and a recess. The safety coupling is formed to fit around the body of an endoscope. Hinges 33 are located on the opposite side of the safety coupling 30 shown in FIGS. 2, 3, 3A, and 4. The safety coupling 30 may have one, two, or more hinges. There also may be embodiments without hinges having a jointed device or flexible piece on which the sides of the safety coupling 30 moves. The safety coupling 30 can be from about 0.5 cm to about 3 cm in length or any combination or subcombination thereof. The overall size of the clamp and endoscope is dependent on the size of the patient's GI tract. The safety coupling is sized to smoothly travail the tract without being impeded.

Another embodiment of the safety system provides a safety coupling 30 as shown in FIG. 4 that comprises channels 31 and 32 in the walls of the safety coupling and auxiliary access channels 41 and 42 on the periphery of the safety coupling 30. The latter being used to accommodate auxiliary endoscopic instruments to be advanced therethrough. Such instruments include forceps, snares, or any other instrument used to contact the body cavity for performing biopsy or other similar functions. In the embodiment illustrated in FIG. 4, a portion of the wall has been thickened to accommodate the larger diameter auxiliary access channels 41 and 42. There are also embodiments comprising at least one auxiliary access channel or at least three.

The safety coupling 30 is adapted to fit snugly around the circumference of an endoscope 50. The inner surface of the safety coupling 30 can comprise an elastomeric material to increase friction between the endoscope 50 and the safety coupling 30. The inner surface of the safety coupling 30 can be internally ridged or textured to assist in preventing movement of the safety coupling 30. The safety coupling 30 has a diameter slightly larger than the diameter of the endoscope 50 body sufficient to provide a secure fit and to prevent the clamp 30 from sliding. In other embodiments, the safety coupling diameter is slightly smaller than the diameter of the ridge 60 positioned distal to the safety coupling 30 such that the ridge 60 prevents distal movement of the safety coupling 30. The ridge 60 can be integrated into an endoscope 50 and/or specifically made for use with the securing systems described herein. For the securing systems used on other endoscopes, the safety coupling 30 fit can be secure enough to prevent distal movement. To further prevent distal movement, one or more elastomeric bands can be placed along the length of the endoscope to maintain the tethers.

As depicted in FIG. 5, the safety harness of the present invention can be a securing sleeve 70. The securing system may comprise a securing sleeve 70 that extends from the cap member 20 in a proximal direction. Such an embodiment is installed by unrolling the tubular securing sleeve 70 over the distal end of the endoscope 50. The sleeve 70, preferably, and in some embodiments, the securing tether, has an inner surface that provides frictional engagement with the endoscope. Such embodiments may or may not have a clamp as the friction fit is sufficient to hold the cap member 20 in place. The securing sleeve 70 or tether can made of an elastomeric material, for example, polyurethane-based elastomer, polyester-based elastomer, polyolefine-based elastomer, polyamide-based elastomer, polystyrene-based elastomer, fluorine-based elastomer, silicone rubber, fluororubber, latex rubber, and the like. They can be used alone or as a mixture of two or more thereof.

An alternate means of securing the cap member includes a helical securing tether configured for attachment to an endoscope. The distal end of the securing tether is attached to the cap member and has a fixed diameter. The diameter is adjustable in a proximal direction along the tether. The helical securing tether wraps around the endoscope and conforms to the endoscope's outer diameter with sufficient gripping force to secure the cap member thereto. The securing tether forms a spiral configured to wind about an endoscope with frictional engagement. When the securing tether is wrapped around an endoscope having a diameter that approximates the diameter of the distal end of the tether, the number of turns decreases. The number of turns increases when the securing tether adjusts to fit around an endoscope having a diameter that is less than the diameter of the distal end of the securing tether. This securing tether can be comprised of an elastomeric polymer or wire.

Generally, attachment of the endoscopic securing system to an endoscope is a method of securing a cap member to the distal end of an endoscope. One method comprises the steps of attaching a cap member to the distal end of the endoscope; providing a safety coupling attaching the safety coupling to an endoscope; and coupling the safety coupling to the cap member using at least one safety line. Another embodiment comprises providing a cap member disposed on the distal end of an endoscope, at least one safety line, and a safety coupling. The safety coupling is secured to the endoscope at a location proximal to and apart from the cap member. At least one tether is coupled between the cap member and the safety coupling. This coupling can be done by threading the line through a channel in the cap member and securing the line in the channel or at the proximal opening of the channel by a knot. Other ways to couple the tether to the cap member include by adhesive or molded into the safety coupling or cap member. The safety coupling is attached to the endoscope as well as the cap member. In some embodiments, there is a ridge distally adjacent to the safety coupling that prevents the safety coupling from moving distally toward the cap member. In other embodiments the cap member is disposed on the distal end of the endoscope and the safety coupling is disposed about the endoscope proximal of and apart from the cap member.

Also provided herein are methods of retrieving a cap member that becomes dislodged from an endoscope using the endoscopic securing systems of the present invention. When a cap member that is part of the present system becomes separated from an endoscope, the cap member remains in contact with the safety coupling by way of at least one safety line. In embodiments where the tethers terminate and connect to the safety coupling, an operator can pull the endoscope out of the body to retrieve the cap member. This eliminates the need to use forceps to retrieve the device. In those embodiments where the tethers extend through the safety coupling to an actuation device, the cap member can be pulled proximally by actuation of the tethers to prevent the cap member from becoming lost in the body. In some instances, an operator can be capable of re-securing the cap member onto the endoscope.

It is therefore intended that the foregoing detailed description be regarded as illustrative rather than limiting, and that it be understood that it is the following claims, including all equivalents, that are intended to define the spirit and scope of this invention.

1. An endoscopic securing system comprising a cap member configured to be disposed on the distal end of an endoscope, a safety harness, and a safety coupling being securable to the endoscope between the proximal end of the endoscope and the cap member, the safety coupling formed to fit around the endoscope, and the safety harness connected to the cap member and extending in a proximal direction to connect with the safety coupling.

2. The system of claim 1 wherein the safety harness comprises one of at least one tether or a securing sleeve.

3. The system of claim 1 wherein the cap member is a ligating barrel or endoscope hood.

4. The system of claim 1 further comprising an endoscope with a ridge spaced apart from a distal end thereof, wherein the safety coupling is disposed about the endoscope proximal of the ridge so as to prevent the safety coupling from moving distally toward a distal end of the endoscope.

5. The system of claim 1 further comprising an endoscope, wherein the cap member is disposed on the distal end of the endoscope and the safety coupling is disposed about the endoscope proximal of and apart from the cap member.

6. The system of claim 1 wherein the safety coupling further comprises at least one auxiliary access channel on the periphery of the safety coupling to accommodate forceps or other instruments to contact the body cavity.

7. The system of claim 1 further comprising an actuation device to which the safety harness is connected.

8. The system of claim 2 wherein the securing sleeve covers a portion of the endoscope from the cap member and extends over and is in frictional engagement with the endoscope.

9. The system of claim 6 wherein the cap member is a ligating barrel and the system further comprises one or more ligating bands removably disposed on the ligating barrel.

10. The system of claim 2 wherein the at least one tether is affixed to the safety coupling.

11. The system of claim 2 comprising at least two tethers.

12. The system of claim 2 wherein the system is mounted on an endoscope and the tether extends through the safety coupling to an actuation device located near a proximal end of the endoscope.

13. The system of claim 2 wherein the safety coupling comprises access channels through which the tether extends.

14. An endoscopic securing system comprising a cap member configured to be disposed on the distal end of an endoscope and a tubular safety harness connected to the cap member for securing the cap member to the distal end of the endoscope member, the safety harness being deployed over the endoscope, providing a friction fit with the endoscope, and extending in a proximal direction from the cap member.

15. The system of claim 14 wherein the safety harness is a securing tether or securing sleeve.

16. The system of claim 14 wherein the securing tether forms a spiral configured to wind about an endoscope with frictional engagement.

17. The system of claim 14 wherein the securing sleeve covers a portion of the endoscope from the cap member and extends over and is in frictional engagement with the endoscope.

18. The system of claim 14 wherein the safety harness is comprised of an elastomeric material.

19. A method of securing a cap member to an endoscope comprising:
providing a cap member disposed on the distal end of an endoscope, a safety harness coupled to the cap member and extending in a proximal direction from the cap member and coupled to a safety coupling;
securing the safety coupling to the endoscope at a location proximal to and apart from the cap member; and
extending the safety harness from the cap member in a proximal direction approaching the safety coupling.

20. The method of claim 19 wherein the safety harness comprises one of at least one tether or a securing sleeve.

21. The method of claim 19 wherein the safety coupling further comprises at least one auxiliary access channel on the periphery of the safety coupling to accommodate forceps or other instruments to be advanced therethrough.

22. The method of claim 19 wherein the cap member is a ligating barrel and further comprises one or more ligating bands removably disposed on the ligating barrel.

23. The method of claim 19 wherein the endoscope further comprises a ridge distally adjacent to the safety coupling that prevents the safety coupling from moving distally toward the cap member.

24. The method of claim 19 wherein the safety coupling comprises access channels through which the at least one tether extends.

25. The method of claim 20 wherein the securing sleeve extends in a proximal direction having a frictional engagement with the endoscope.
